(19) United States
(12) Patent Application Publication
(10) Pub. No.: US 2020/0277210 A1
(43) Pub. Date: Sep. 3, 2020

(54) PROCESS AND APPARATUS FOR IN-SITU CLEANING OF A GAS SEPARATOR IN AN ANAEROBIC BIOREACTOR

(71) Applicant: Veolia Water Solutions & Technologies Support, Saint Mauricef (FR)

(72) Inventor: Robertus Johannes FRANKIN, Delft (NL)

(73) Assignee: Veolia Water Solutions & Technologies Support, Saint Maurice (FR)

(21) Appl. No.: 16/068,069

(22) PCT Filed: Jan. 6, 2016
(86) PCT No.: PCT/EP2016/050114

§ 371 Date: Jul. 3, 2018

(51) Int. Cl. C02F 3/28; C02F 11/04; C12M 1/107; C12M 1/00

(52) U.S. Cl. CPC C02F 3/2846; C02F 11/04; C12M 21/04; C02F 2303/20; C12M 39/00; C02F 2301/024; C02F 2203/006; C12M 29/24

(57) ABSTRACT

The present invention relates to a process for in-situ cleaning of a gas-liquid separator of an anaerobic bioreactor, comprising directing a gas flow in the bioreactor in order to create a scouring effect from turbulent fluid flows resulting in the cleaning of at least a part of the gas-liquid separator. The invention further relates to a process for treating a fluid aqueous waste stream, wherein use is made of the in-situ cleaning process. The invention further relates to a bioreactor suitable for carrying out a process according to the invention.

The invention relates to a process for in-situ cleaning of a gas-liquid separator of an anaerobic bioreactor, to a process for treating a fluid aqueous waste stream, and to an apparatus (bioreactor) suitable for said in-situ cleaning processes.

Biological treatment of a waste stream uses active biomass (bacteria) to degrade biodegradable pollutants (biodegradable organic substance) in the waste stream, for instance a fluid aqueous waste stream.

For so-called anaerobic treatment (without oxygen) a consortia of anaerobic bacteria, which are generally known in the art, convert pollutants substantially to biogas, which is typically rich in methane. These anaerobic bacteria mainly grow in aggregates, often referred to as granular biomass. Under anaerobic conditions, the production of surplus sludge (new biomass (bacteria) as a result of bacterial growth) is generally relatively low, because typically only a small part of the biodegradable substance in the waste is used by the bacteria for bacterial growth.

Suitably, the treatment of a fluid aqueous waste stream comprises feeding the aqueous waste stream into the lower part of a bioreactor containing granular biomass thereby producing biogas in the treatment and passing the resulting gas/liquid/solid mixture upward and separating the gas from the liquid phase in a gas-liquid separator.

Such gas-liquid separators are used in many different bioreactor systems known in the art.

However, it is a known problem in the art that such gas-liquid separators can suffer from blockages and encrustation due to the accumulation of solids, such as biomass, in the baffle plate arrangements or the like, typically present in such gas-liquid separators. These blockages lead to decreased performance of these bioreactor systems due to unequal water distributions and local high velocities in the gas liquid separators which will disturb the settling of solids back into the reactor vessel of the bioreactor. Present processes for removing these (biomass) blockages from such gas-liquid separators require that the bioreactors are shut down and taken out of operation in order to open up the bioreactors to clean the gas-liquid separators. This is disadvantageous since it leads to significant down time, which is not only economically prohibitive but also has associated risks from a health and safety perspective.

WO 2007/078195 A1 describes a process and a reactor for the anaerobic purification of waste water using a sludge bed system, which process comprises feeding waste water, and optionally recycle water, to the lower part of an upflow reactor, containing mainly granular biomass thus producing biogas in the treatment passing the resulting gas/liquid/solid mixture upward and separating the gas and solid from the liquid in a three phase separator and thereby generating an anaerobic effluent flow that is withdrawn from the top of the separator. WO 2007/078195 A1 also describes that the three phase separator contains an anaerobic effluent recirculation collection pipe at the bottom, with several openings/slots, which anaerobic effluent extraction provides the possibility for in-process cleaning of the three phase separator and its internals by the introduction of a back flow of water or bio (gas) recycles through the same pipe and holes or slots.

A disadvantage of known methods for cleaning a gas-liquid separator in an anaerobic bioreactor is that additional pumps or blowers are required to direct fluid flow into the gas-liquid separator to clean it. These pumps or blowers not only increase the economic cost of the bioreactor itself, but also result in a loss of productivity as typically the treatment of a waste water stream cannot be continued while the cleaning process is carried out.

An object of the invention is to provide an alternative process, in particular an improved process for cleaning a gas-liquid separator in a bioreactor. A further object is to provide a bioreactor suitable for anaerobic treatment of an aqueous waste stream that can be cleaned using a cleaning process according to the invention.

Surprisingly this object is achieved by using a process for in-situ cleaning of a gas-liquid separator of an anaerobic bioreactor, to a process for treating a fluid aqueous waste stream, and a bioreactor suitable for said processes with a specific piping arrangement whereby gas is directed in the bioreactor in a specific way.

Accordingly, in a first aspect the invention relates to a process for in-situ cleaning of a gas-liquid separator of an anaerobic bioreactor, comprising directing a gas flow in the bioreactor in order to create a scouring effect from turbulent fluid flows resulting in the cleaning of at least a part of the gas-liquid separator.

In a second aspect, the invention relates to a process for treating a fluid aqueous waste stream comprising a biodegradable organic substance, comprising:
feeding the aqueous waste stream into an anaerobic bioreactor;
reacting the biodegradable organic substance with the biomass in the bioreactor under essentially anaerobic conditions, thereby forming biogas (methane);
said process comprising carrying out a process for in-situ cleaning according to the first aspect of the invention, whilst continuing the treatment of the waste stream.

In a third aspect, the invention relates to a bioreactor suitable for the process of invention, wherein said bioreactor comprises:
a reactor vessel for containing at least a fluid (such as a gas liquid mixture);
an influent inlet for introducing a flow of a fluid aqueous waste stream comprising a biodegradable organic substance into the reactor vessel;
an effluent outlet for withdrawing an anaerobic effluent flow from the reactor vessel;
a gas outlet for withdrawing gas from the reactor vessel;
a gas-liquid separator present in the reactor vessel, wherein the gas-liquid separator comprises a gas collector and a gas channel; and,
a gas pipe which is connected to the gas-liquid separator, which gas pipe is further adapted to allow the passage of a gas from the gas channel to a headspace of the reactor vessel, or, is fluidly connected to an open-ended chamber of the reactor vessel.

Such process is typically used to result in a purified aqueous stream and provide biogas as a source for methane, which can be flared off or used to produce energy.

An advantage of the invention is that the in-situ cleaning process requires no down time and can be carried out in an anaerobic bioreactor system, whilst the anaerobic treatment process of a waste stream is continued. A process according to the invention may be suitable used in bioreactors which carry out the anaerobic treatment process in a continuous or a batch process. Another advantage is that no additional pumps or blowers are required to be installed in the bioreactor to carry out a cleaning process according to the invention. In addition, the biogas produced in the bioreactor can be advantageously used in the cleaning process without requiring an additional internal distribution arrangement inside the reactor vessel. A further advantage is that the processes can be carried out in an ad-hoc manner (i.e. carried out for a particular purpose as necessary), in the case of reduced bioreactor performance being attributed to a suspected (biomass) blockage. Alternatively or additionally, the process can be automated to be carried out periodically so as to advantageously provide preventative cleaning whereby excessive buildup of solids (such as biomass) is at least substantially avoided.

In a first preferred process for in-situ cleaning of a gas-liquid separator of an anaerobic bioreactor, the gas-liquid separator comprises a gas collector which is fluidly connected to a closable gas pipe, which gas pipe is further fluidly connected to an open-ended chamber of the reactor vessel, which in-situ cleaning process comprises a step of closing the gas pipe, thereby allowing gas to release from underneath the gas collector, thereby causing the scouring effect from a turbulent fluid flow resulting in the cleaning of at least a part of the gas-liquid separator.

Surprisingly, this first preferred embodiment may be advantageously used to remove substantial (biomass) blockages in the gas-liquid separator without requiring the complete shutdown of the bioreactor. Substantial (biomass) blockages in the gas-liquid separator can result in the gas-liquid separator becoming inoperable due to obstructions caused by these blockages, which is usually indicated by substantially reduced efficiency in the removal of biodegradable organic substances from the waste stream and in the associated biogas production. Such partial (biomass) blockages typically result in a short circuiting in the anaerobic treatment process and dead space in the bioreactor, which is usually indicated by reduced efficiency in the removal of biodegradable organic substances from the waste stream, less biogas production, or by cold spots on the bioreactor walls.

In a second preferred process for in-situ cleaning of a gas-liquid separator of an anaerobic bioreactor, the gas-liquid separator comprises a gas channel, which gas channel is provided with a closable gas pipe adapted to allow passage of a gas from the gas channel to a headspace of the reactor vessel,

which in-situ cleaning process comprises opening the gas pipe, thereby allowing gas to release into the headspace, thereby causing a scouring effect from a turbulent fluid flow resulting in the cleaning of at least a part of the gas channel and/or the gas pipe.

Surprisingly, this second preferred cleaning process of the invention may be advantageously used to remove partial (biomass) blockages in the gas channel or gas pipe. In this case, said process may be carried out more quickly than the process of the first preferred embodiment of the cleaning process the invention since less gas is required for said in-situ cleaning process. A further advantage of this preferred embodiment, is that it allows the cleaning out of the gas channel and the gas pipe without obstructing (i.e. minimal impact on) the normal operation of the bioreactor system.

A further advantage of the process for in-situ cleaning according to the invention is that it allows the gas-fluid interface level inside the gas-liquid separator or gas channel to be manipulated by the closing or opening of the gas pipe. This leads to the lowering or raising of the gas-fluid interface level inside the gas-liquid separator or gas channel, thereby causing turbulent fluid flow essentially only in these sections of the reactor vessel and thus these in-situ cleaning processes have limited impact on the treatment process carried out the reactor vessel.

The term “or” as used herein is defined as “and/or” unless specified otherwise.

The term “a” or “an” as used herein is defined as “at least one” unless specified otherwise, or it follows from the context that it should refer to the singular only.

When referring to a noun (e.g. a compound, an additive, etc.) in the singular, the plural is meant to be included, or it follows from the context that it should refer to the singular only.

As used herein “biodegradable organic substance” is an organic substance that can be converted by biomass in the reactor under essentially anaerobic conditions, in particular into biomass or methane.

As used herein “organic substance” is any organic substance that is chemically oxidisable, as can be determined by the Chemical Oxygen Demand (COD) test, as described in ISO 6060:1989.

The term “normal operation” as used herein is defined as the anaerobic treatment process of a waste stream carried out in an anaerobic bioreactor while a cleaning step is not carried out (thus, before, after or in between cleaning with a cleaning process according to the invention).

The term “biogas” as used herein is defined as an in-situ product of the process for treating a fluid aqueous waste stream carried out in a bioreactor, which is typically rich in methane.

The term “closable” as used herein is defined as being reversibly closable, unless specified otherwise.

The term “blockage” as used herein refers to blockages, debris, encrustation and buildup/accumulation of solids, such as dirt and biomass.

The term “bioreactor” as used herein refers to an anaerobic bioreactor.

The term “substantial(ly)” or “essential(ly)” is generally used herein to indicate that it has the general character or function of that which is specified. When referring to a quantifiable feature, these terms are in particular used to indicate that it is for at least 75%, more in particular at least 90%, even more in particular at least 95% of the maximum that feature.

The term ‘essentially free’ is generally used herein to indicate that a substance is not present (below the detection limit achievable with analytical technology as available on the effective filing date) or present in such a low amount that it does not significantly affect the property of the product that is essentially free of said substance. In practice, in quantitative terms, a product is usually considered essentially free of a substance, if the content of the substance is 0-0.1 wt. %, in particular 0-0.01 wt. %, more in particular 0-0.001 wt. %.

The turbulent fluid flow allows the scouring/removal of blockages, debris, encrustation and buildup of solids, such as dirt and biomass present in or on at least part of the gas-liquid separator or gas channel and gas pipe of the bioreactor.

For the purpose of clarity and a concise description features are described herein as part of the same or separate embodiments, however, it will be appreciated that the scope of the invention may include embodiments having combinations of all or some of the features described.

In an advantageous embodiment, the gas used in the cleaning processes of the invention essentially consists of biogas produced in-situ by the anaerobic conversion of an organic substance in the bioreactor. Optionally, said gas used for cleaning further comprises an external gas source (typically low in oxygen (i.e. <1 vol. % oxygen), and preferably essentially free of oxygen), in particular an external gas source selected from the group consisting of methane and nitrogen.

The gas-liquid separator used in the processes and bioreactor of the invention can be those known in the art. Typical examples of gas-liquid separators include three phase settlers, (internal) settlers and baffle (plate) arrangements. Such gas-liquid separators guide the gas away from a zone where quiescent conditions allow the (biomass) solids to settle and return to the main reactor vessel body. Usually the gas-liquid separator is present at least partly submerged in the fluid in the reactor vessel. The gas-liquid separator typically has an opening in the lower extremity which is fluidly connected to the reactor vessel body (for transporting fluid from the reactor vessel into the gas-liquid separator), and an opening in the upper extremity which is fluidly connected to the gas collector (for transporting gas from the gas-liquid separator to the gas collector).

Preferably, the bioreactor comprises a plurality of gas-liquid separators. In an embodiment, the gas-liquid separators are arranged in a single layer. In a further embodiment, the gas-liquid separators are arranged in multiple (two or more), preferably staggered, levels in the reactor vessel of the bioreactor. In particular for a reactor design with multiple levels of gas-liquid separators, the cleaning of the lower level(s) is largely facilitated with a cleaning process according to the invention, because these are difficult, if not practically impossible, to access from outside the reactor.

Preferably, the gas-liquid separators are arranged in in the upper part (upper half) of the reactor vessel.

The gas collector used in the processes and the bioreactor of the invention is typically a gas hood or the like. Typically, the gas collector is present in the upper part of the gas-liquid separator, which gas collector has an opening in the lower extremity which is fluidly connected to the gas-liquid separator (for transporting gas from the gas-liquid separator to the gas collector). The gas collector usually further has an opening in the upper extremity which is fluidly connected to the gas channel or gas pipe (for withdrawing gas from the gas collector).

A suitable gas channel which may be used in the gas-liquid separator in the processes and bioreactor of the invention is a gas box or gas piping. In the case that the gas channel is a gas piping, the collected gas is usually withdrawn directly from the top of the gas collector. Typically, the gas channel is present in the reactor vessel adjacent to the gas-liquid separator and below the gas pipe. The gas channel is usually provided with at least one opening for introducing gas into the gas channel and another opening for transporting gas in the gas channel into the gas pipe.

Typically, the gas pipe comprises an inlet and an outlet. In a preferred embodiment, the inlet of the gas pipe is fluidly connected to the gas collector and the outlet of the gas pipe is fluidly connected to the open-ended chamber of the reactor vessel. In a further preferred embodiment, the inlet of the gas pipe is connected to the gas channel and the outlet of the gas pipe is adapted to allow passage of a gas from the gas channel to the headspace of the reactor vessel.

The gas pipe may be a branched gas pipe, having at least two branches each providing an outlet, wherein a first gas pipe branch is connected (via the outlet) to a different part of the reactor vessel than the second gas pipe branch. Preferably, at least one gas pipe branch is adapted to allow passage of the gas from the gas pipe to the headspace of the reactor vessel whereas the second gas pipe is connected to allow passage of the gas to the open-ended chamber of the reactor vessel.

The gas pipe may be suitably reversibly closable by one or more valves or other closing means present in or at an end of the said gas pipe. Preferably, the one or more valves or other closing means present in or at an end of the said gas pipe are also present outside of the bioreactor, which has the advantage that said means and valves are more easily accessible and allow for greater control of the gas-fluid interface level in the gas channel and gas-liquid separator.

An operator will be able to determine when it is desirable to commence a cleaning process according to the invention based on the information disclosed herein and common general knowledge.

One may determine on a case to case basis to commence the cleaning process, in particular on the basis of a deviance in relevant operating parameters, such as a reduced efficiency in the removal of biodegradable organic substances from the waste stream, less biogas production, and/or by cold spots on the bioreactor walls. This can be done manually or in an automated manner, i.e. by automatically monitoring one or more parameters and automatically initiate the cleaning process when parameters of choice are outside a pre-set range.

A preferred bioreactor system that is thus operated in an automatic manner is provided with a measurement device for monitoring one of more of said parameters having a signal output, a controller having a receiver for said signal output, the controller further having a function for determining whether the one or more parameters are within the pre-set range or not, and an output to activate and stop the cleaning process (an open/close signal to the opening means in the gas pipe or gas pipes).

In a further preferred embodiment, the gas pipe is closed and re-opened intermittently (at periodic intervals, such as cleaning at least once a week, at least once a fortnight, or at least once a month), thereby providing a preventative cleaning process whereby excessive build up of solids (such as biomass) is at least substantially avoided. In particular in such embodiment, the one or more valves or other closing means are provided with a controller, such as a timer for setting the intervals for the periodic opening and closing of the gas pipe. The skilled person will be able to determine an appropriate interval for carrying out the in-situ cleaning process of the invention, based on the information disclosed herein and common general knowledge.

Typically, under normal operating conditions of a bioreactor, the valve of a closable gas pipe adapted to allow passage of a gas from the gas channel to the headspace of the reactor vessel is closed.

The open-ended chamber of the reaction vessel is typically present in the reactor vessel adjacent to the gas-liquid separator. Usually, the open-ended chamber of the reaction vessel has a higher gas-fluid interface level than the rest of the reactor vessel due to gas holdup, which is a result of gas being introduced by the outlet of the gas pipe into the fluid contained in said chamber.

Typically the anaerobic effluent flow (cleaned effluent) is withdrawn from the reactor vessel through an effluent outlet, such as an effluent discharge pipe, open launder or any other means, preferably from the upper part of the reactor vessel, above the gas-liquid separator.

(Bio)gas is usually withdrawn from the reactor vessel through a gas (effluent) outlet, such as a closable gas discharge pipe, preferably from the headspace of the reactor vessel.

The influent inlet for introducing a flow of a fluid aqueous waste stream into the reactor vessel may suitably be an influent distribution system. Preferably, the influent distribution system is present in a lower part of the reactor vessel.

A process of the invention may suitably be carried out in bioreactor-types known in the art, such as upflow reactors, with the provision that they are provided with one or more of the gas pipes to direct a gas flow, as described herein. Preferably, the bioreactor to be used in the process of the invention is selected from the group consisting of upflow anaerobic sludge blanket reactors (UASB), expanded granular sludge blanket reactors (EGSB), internal circulation reactors (IC), fluidized bed reactors, anaerobic baffled reactors and anaerobic filters.

Typically, the reactor vessel contains a fluid which is a gas-liquid mixture produced by the anaerobic treatment of a waste stream. The fluid also usually contains biomass and a biodegradable organic substance that is at least partially converted by the biomass under essentially anaerobic conditions, thereby forming biogas (such as methane).

The various aspects of the invention are now illustrated on the basis of the Figures, wherein the gas-liquid separators are provided in two levels, of which one level is optional.

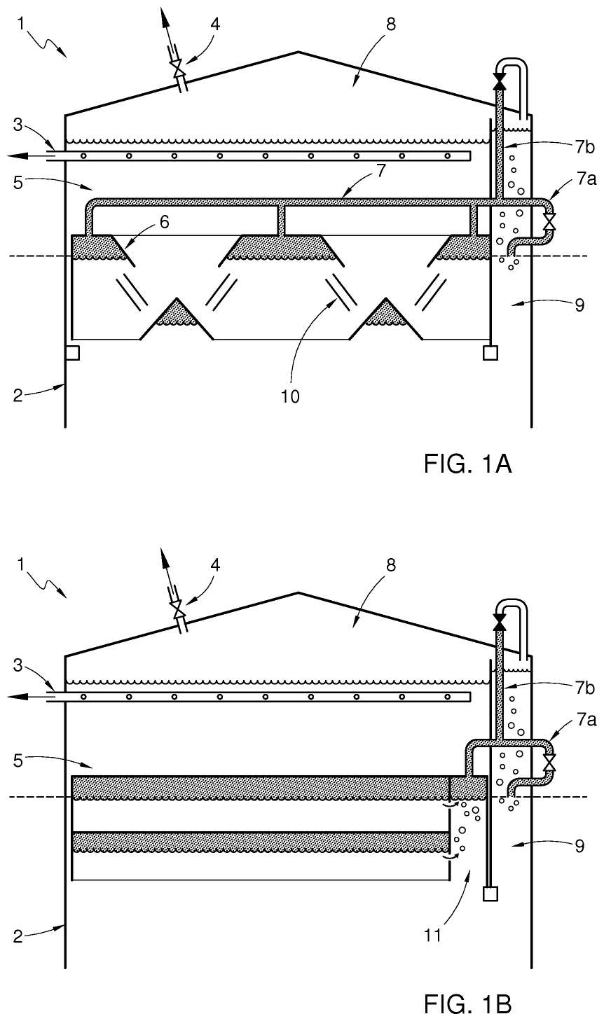

FIGS. 1a and 1b show different cross sectional views, which are 90 degrees horizontally rotated with respect to each other, of a bioreactor and gas-liquid separator in normal operation,

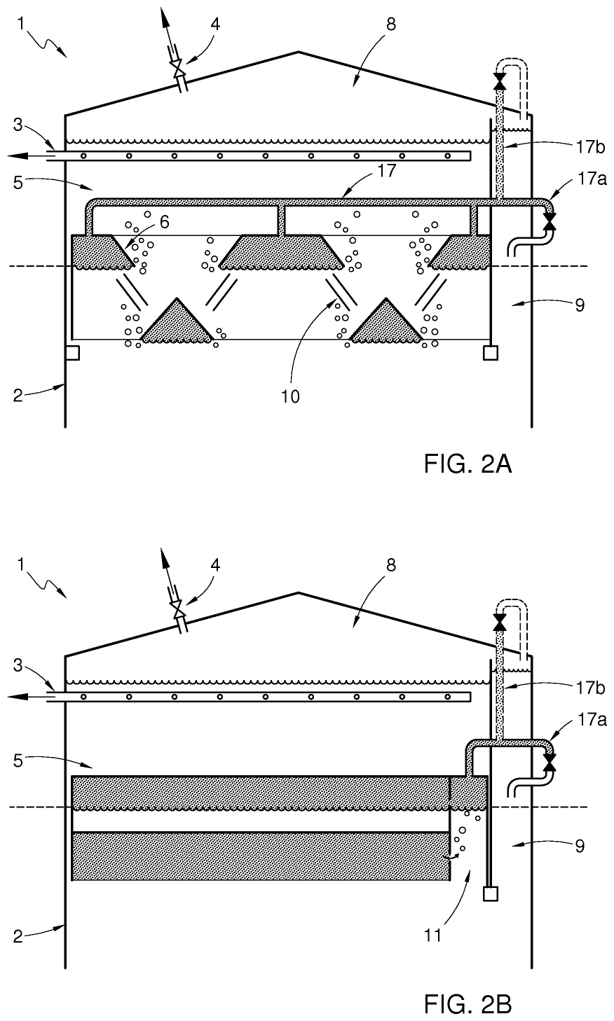

FIGS. 2a and 2b show different cross sectional views, which are approximately 90 degrees horizontally rotated with respect to each other, of a bioreactor in the cleaning mode for the gas-liquid separator (corresponding to the first aspect of the invention and first preferred embodiment),

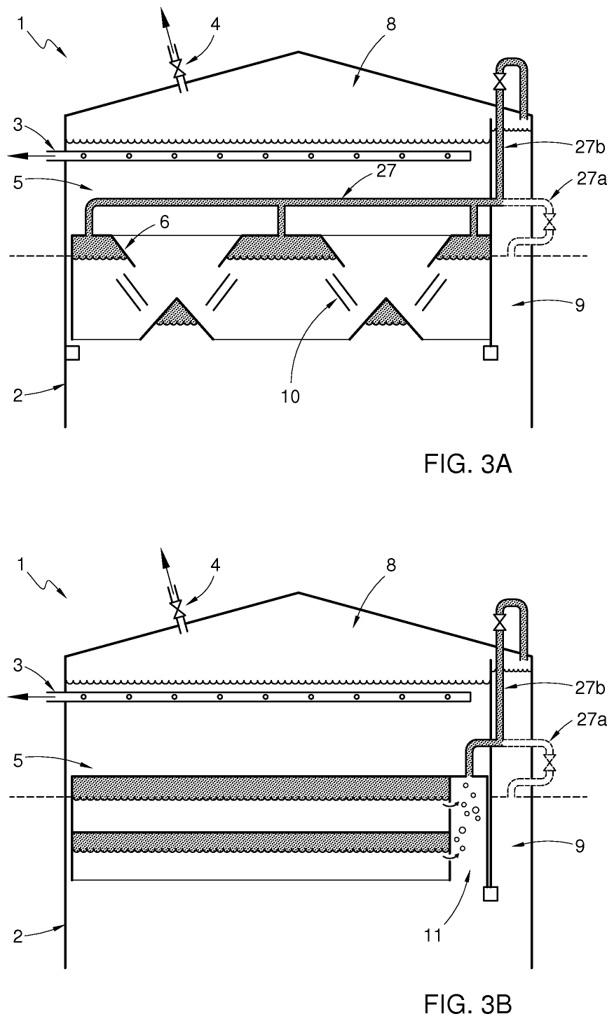

FIGS. 3a and 3b show different cross sectional views, which are approximately 90 degrees horizontally rotated with respect to each other, of a bioreactor in cleaning mode for the gas channel and first gas pipe of the bioreactor (corresponding to the first aspect of the invention and the second preferred embodiment).

FIGS. 1a and 1b show a bioreactor (1) according to the invention in normal operation, which bioreactor (1) comprises the reactor vessel (2) containing a fluid aqueous waste water which is fed into the reactor vessel via an inlet (not shown). The waste water rises in the reactor vessel (2), wherein a sludge bed is present consisting of mainly granular sludge. Due to the anaerobic breakdown of the (biological) contaminants in the waste water biogas (such as methane) is formed and a mixture of solid, liquid and gas develops. The fluid, such as a gas-liquid mixture, passes (flows) upwards and enters the submerged gas-liquid separator (5), where the gas is separated from the mixture via a titled baffle (plate) arrangement (10). The separated gas is collected by the gas collectors (gas hoods) (6), which gas is indicated by the dotted shading in FIGS. 1a and 1b. The collected gas is then transported via the gas channel (11) to the branched gas pipe (7, 7a, 7b). The valve on the gas pipe (7b) is closed, while the valve on the gas pipe (7a) is open, which allows the gas to escape via the outlet of the gas pipe (7a) into the fluid contained in the open-ended chamber (9) of the reactor vessel (2). This enables the gas-fluid interface level inside the gas-liquid separator (5) to be maintained. The gas released into the open-ended chamber (9) causes an in the fluid level in the open-ended chamber (9) of the reactor vessel (2), since the fluid becomes less dense. An anaerobic effluent flow (cleaned effluent) is withdrawn through the effluent discharge pipe (3) present in the upper part of the reactor vessel (2). The (bio)gas produced is removed from the headspace (8) of the reactor vessel (2) via the gas discharge pipe (4) present at the top of the bioreactor (1).

FIGS. 2a and 2b show a bioreactor (1) in cleaning mode for the gas-liquid separator (5) of the bioreactor (2). The difference between FIGS. 1a and 1b and FIGS. 2a and 2b, is that in FIGS. 2a and 2b the valve of the gas pipe (17,17a) is closed. Optionally, the gas pipe (17,17a) comprises a gas pipe branch (17b) indicated by a dashed line, which gas pipe branch (17b) also has a closed valve. By closing the valve(s) of the gas pipe (17,17a,17b), gas is prevented from escaping into the open-ended chamber (9) of the reactor vessel (2) and the gas pressure builds up underneath the gas collector (gas hoods) (6). This gas pressure build-up continues until the pressure is sufficient to lower the gas-fluid interface level in the gas-liquid separator (5) leading to the gas escaping causing a turbulent fluid flow resulting in the cleaning of at least part of the gas-liquid separator (5).

FIGS. 3a and 3b show a bioreactor (1) in cleaning mode for the gas channel (11) and gas pipe (27,27b) of the gas-liquid separator (5) of the bioreactor (1). The difference between FIGS. 1a and 1b and FIGS. 3a and 3b, is that in FIGS. 3a and 3b, the valve of the gas pipe (27,27b) is open, which gas pipe (27,27b) has an outlet fluidly connected to the head space (8) of the reactor vessel (2). The gas pipe (27,27b) optionally comprises gas pipe branch (27a) indicated by the dashed line, which gas pipe branch (27a) is fluidly connected the open-ended chamber (9) of the reactor vessel (2) via an open valve. By opening the valve(s) of the gas pipe (27,27b,27a), the gas is allowed to escape directly upwards to the headspace (8) of the bioreactor (1) and this causes the gas-fluid interface level in the gas channel (11) to rise, thereby causing a turbulent fluid flow resulting in the cleaning of at least part of the gas channel (11) and the gas pipe (27,27b,27a).

1. A process for in-situ cleaning of a gas-liquid separator of an anaerobic bioreactor, comprising directing a gas flow in the bioreactor in order to create a scouring effect from turbulent fluid flows resulting in the cleaning of at least a part of the gas-liquid separator.

2. The process of claim 1, wherein the gas-liquid separator comprises a gas collector which is fluidly connected to a closable gas pipe, which gas pipe is further fluidly connected to an open-ended chamber of the reactor vessel,
which in-situ cleaning process comprises a step of closing the gas pipe, thereby allowing gas to release from underneath the gas collector, thereby causing the scouring effect from a turbulent fluid flow resulting in the cleaning of at least a part of the gas-liquid separator.

3. The process of claim 1, wherein the gas-liquid separator comprises a gas channel, which gas channel is provided with a closable gas pipe adapted to allow passage of a gas from the gas channel to a headspace of the reactor vessel,
which in-situ cleaning process comprises opening the gas pipe, thereby allowing gas to release into the headspace, thereby causing a scouring effect from a turbulent fluid flow resulting in the cleaning of at least a part of the gas channel and the gas pipe.

4. A process for treating a fluid aqueous waste stream comprising a biodegradable organic substance, comprising
feeding the aqueous waste stream into an anaerobic bioreactor;
reacting the biodegradable organic substance with the biomass in the bioreactor under essentially anaerobic conditions, thereby forming biogas;
said process comprising carrying out a process for the in-situ cleaning claim 1, whilst continuing the treatment of the waste stream.

5. The process of claim 1, wherein the gas pipe is a branched pipe having at least two gas outlets, connected to different parts of the reactor vessel.

6. The process of claim 5, wherein at least one gas pipe branch is adapted to allow passage of the gas from the gas pipe to the headspace of the reactor vessel and the second gas pipe branch is connected to the open-ended chamber of the reactor vessel.

7. The process of claim 1, wherein the gas pipe is reversibly closable by one or more valves or other closing means present in or at an end of the said gas pipe.

8. The process of claim 1, wherein the gas used in said cleaning process essentially consists of biogas produced by the anaerobic conversion of the organic substance in the bioreactor.

9. The process of claim 1, wherein the gas used in said cleaning process comprises biogas and further comprises an external gas source.

10. The process of claim 1, wherein the bioreactor comprises a plurality of gas-liquid separators, wherein the gas-liquid separators are arranged in multiple staggered levels in the reactor vessel of the bioreactor.

11. The process of claim 1, wherein the gas pipe is automatically closable and openable.

12. The process of claim 1, wherein the gas pipe is closed and re-opened intermittently, thereby providing a preventative cleaning whereby excessive buildup of solids is at least substantially avoided.

13. The process of claim 1, wherein the bioreactor is an upflow reactor.

14. A bioreactor suitable for the process of claim 1, wherein said bioreactor comprises:
a reactor vessel for containing at least a fluid;
an influent inlet for introducing a flow of a fluid aqueous waste stream comprising a biodegradable organic substance into the reactor vessel;
an effluent outlet for withdrawing an aqueous effluent flow from the reactor vessel;
a gas outlet for withdrawing gas from the reactor vessel;
a gas-liquid separator present in the reactor vessel, wherein the gas-liquid separator comprises a gas collector and a gas channel; and,
a gas pipe which is connected to the gas-liquid separator, which gas pipe is further adapted to allow the passage of a gas from the gas channel to a headspace of the reactor vessel, or, is fluidly connected to an open-ended chamber of the reactor vessel.

15. The bioreactor of claim 14, wherein the gas pipe is a branched pipe having at least two branches with an outlet, wherein the outlet of the first branch is connected to a different part of the reactor vessel than the outlet of the second branch.

16. The bioreactor of claim 15, wherein the first gas pipe branch is adapted to allow passage of the gas from the gas pipe to the headspace of the reactor vessel and wherein the second gas pipe branch is connected to the open-ended chamber of the reactor vessel.

17. The bioreactor of claim 14, wherein the gas pipe is reversibly closable by one or more valves or other closing means present in or at an end of said gas pipe.

18. The bioreactor of claim 14, wherein the closing means of the gas pipe are one or more automatic open/close valves or other closing means.

19. The bioreactor of claim 18, wherein the one or more valves or other closing means are provided with a controller to allow automated intermittently opening and closing the gas pipe.

20. The bioreactor of claim 14, wherein the bioreactor comprises a plurality of gas-liquid separators, which are arranged in multiple staggered levels in the reactor vessel of the bioreactor.

21. The bioreactor of claim 14, wherein the influent inlet for the bioreactor is an influent distribution system.

22. The process of claim 7, wherein said one or more valves or other closing means present in or at an end of the said gas pipe are present outside of the bioreactor.

23. The process of claim 9, wherein the external gas source is selected from the group consisting of methane and nitrogen.

24. The process of claim 13, wherein the upflow reactor is selected from the group consisting of upflow anaerobic sludge blanket reactors, expanded granular sludge blanket reactors, internal circulation reactors, fluidized bed reactors, anaerobic baffled reactors and anaerobic filters.

25. The bioreactor of claim 21, wherein said influent distribution system is present in a lower part of the reaction vessel.
